(19) United States
(12) Patent Application Publication
(10) Pub. No.: US 2017/0322484 A1
(43) Pub. Date: Nov. 9, 2017

(54) X-RAY PRE-EXPOSURE CONTROL DEVICE

(71) Applicant: KONINKLIJKE PHILIPS N.V., EINDHOVEN (NL)

(72) Inventor: KLAUS ERHARD, HAMBURG (DE)

(21) Appl. No.: 15/527,029

(22) PCT Filed: Nov. 16, 2015
(86) PCT No.: PCT/EP2015/076673

§ 371 Date: May 16, 2017

(30) Foreign Application Priority Data
Nov. 19, 2014 (EP) 14193772.2

(51) Int. Cl. G03B 42/02; A61B 6/04; A61B 6/00; G06T 7/136

(52) U.S. Cl. CPC G03B 42/026; A61B 6/04; A61B 6/466; G06T 7/136; A61B 6/462

(57) ABSTRACT

The invention relates to an X-ray pre-exposure control device (10), an X-ray imaging system (1), an X-ray imaging method, and a computer program element for controlling such device and a computer readable medium having stored such computer program element. The X-ray pre-exposure control device (10) comprises a subject detection unit (11), a subject model unit (12), an interface unit (13), a processing unit (14), and a display unit (15). The subject detection unit (11) is configured to detect subject data of the subject (111) to be exposed. The subject model unit (12) is configured to provide a subject model and to refine the subject model based on the subject data into a refined subject model. The interface unit (13) is configured to provide setting data of an X-ray unit (131) to be used for exposing the subject. The processing unit (14) is configured to calculate a virtual X-ray projection (151) based on the refined subject model and the provided setting data. The display unit (15) is configured to display the virtual X-ray projection (151).

### FIELD OF THE INVENTION

The invention relates to an X-ray pre-exposure control device, an X-ray imaging system, an X-ray imaging method, and a computer program element for controlling such device and a computer readable medium having stored such computer program element.

### BACKGROUND OF THE INVENTION

WO 02/093986 A1 discloses an X-ray examination device designed to automatically process one or a series of X-ray examinations. The automatic processing includes the setting of the power of the X-ray equipment, the setting of the parameters of the X-ray table, the type of examination to be performed, and the reporting and archiving functions. The apparatus also takes into account data which is specific of the patient to be examined, for example, the identity, the weight of the body and the part of the body to be examined.

However, in certain cases, after an X-ray examination has been performed, there may be a need to redo the examination. For example, due to patient positioning issues, collimation problems and/or incorrect exposure, the examination result may be unsatisfactory. In particular in chest X-ray (CXR) examination, it is estimated this occurs in 5% of the cases, which could be higher in certain markets where radiographers may be less experienced.

As a result, there is a need to reduce the number of patient positioning issues, collimation problems and/or incorrect exposure to reduce the number of retakes and thereby the X-ray dosage.

US 2012/089377 A1 discloses a method comprising generating, with a processor, a three-dimensional subject specific model of the subject to be scanned based on a general three-dimensional model and pre-scan image data acquired by an imaging system.

US 2006/198499 A1 discloses a method of adapting imaging parameters for a computer tomographic radiograph of a body volume based on a three-dimensional pilot radiograph obtained with a low dose of radiation.

### SUMMARY OF THE INVENTION

Hence, there may be a need to provide an improved X-ray pre-exposure control device, which allows reducing the need to redo X-ray examinations.

The problem of the present invention is solved by the subject-matters of the independent claims, wherein further embodiments are incorporated in the dependent claims. It should be noted that the aspects of the invention described in the following apply also to the X-ray pre-exposure control device, the X-ray imaging system, the X-ray imaging method, the computer program element, and the computer readable medium.

The invention relates to diagnostic imaging, in particular chest X-ray (CXR) examinations. According to the present invention, an X-ray pre-exposure control device is presented. The X-ray pre-exposure control device comprises a subject detection unit, a subject model unit, an interface unit, a processing unit, and a display unit. The subject detection unit is configured to detect subject data of the subject to be exposed. The subject model unit is configured to provide a subject model and to refine the subject model based on the subject data into a refined subject model. The interface unit is configured to provide setting data of an X-ray unit to be used for exposing the subject. The processing unit is configured to calculate a virtual X-ray projection based on the refined subject model and the provided setting data. The display unit is configured to display the virtual X-ray projection.

In other words, the invention proposes calculating or computing a virtual X-ray projection or image based on a subject model of a subject to be exposed and based on current setting data of an X-ray imaging system to be used for exposing the subject. The invention thereby provides e.g. a virtual chest X-ray for pre-exposure position and quality control by e.g. a radiographer prior to an actual X-ray or CXR exposure. A virtual X-ray projection is computed, which can be used to check the positioning of e.g. the X-ray collimators as well as the positioning of the subject to avoid retakes.

As a result, it is proposed to simulate a virtual X-ray projection or image prior to the X-ray exposure in order to verify a correct positioning of the subject, a proper collimation of the X-ray source and/or a suitable exposure. Thereby, the number of repeated X-ray examinations due to patient positioning issues, collimation problems and/or incorrect exposure is reduced. As a consequence, also the dose exposure of the subject, the costs and the amount of time is reduced. As a result, the X-ray pre-exposure control device according to the present invention provides a quality control of subject positions and X-ray settings.

Exemplarily, subject data of the subject to be exposed are detected by tracking a body shape of the subject or patient using for example optical cameras or 3D depth sensors combined with infrared light. The cameras can be integrated in a detector housing. In the resulting subject data or body shape, landmarks (e.g. shoulders, neck, hip bones) can be extracted. The general body shape and size can be used to select, from a database of software models, a most similar subject model (e.g. a thorax model adult male small obese). This selected subject model can be further adjusted or adapted into a refined subject model e.g. by an adaptation to the extracted landmarks. Subsequently, setting data of an X-ray unit to be used for exposing the subject (e.g. focal spot position, position and orientation of X-ray source and X-ray detector, collimator positions, etc.) can be derived from the X-ray unit. Then, from the refined or adapted subject model and the retrieved setting data of the X-ray unit, a simulated or virtual exposure or X-ray projection can be generated or computed in the current view geometry. The virtual X-ray projection can be displayed on a display unit, as e.g. a viewing monitor.

The actual collimated area or window and the virtual X-ray projection can be visualized or displayed to e.g. a radiographer to decide whether e.g. the positioning of the subject and/or the collimation of the X-ray source are suitable for the current examination. The virtual X-ray projection may be larger than an actual field of view. A user interface can be provided to allow an operator to adjust e.g. the position of the collimators with direct visual feedback within the virtual X-ray projection. Thereby, the X-ray pre-exposure control device according to the present invention provides a quality control of position and X-ray settings.

In an example, the subject detection unit is at least one of the group of an optical, a time-of-flight, an infrared, an ultrasound, a radar camera or sensor, a weight sensor, a 3D depth sensor, a sensor sensing a breathing cycle, a sensor sensing a heart cycle, a millimeter wave sensor and a backscatter X-ray sensor. The camera or sensor may be combined with infrared light. The subject detection unit is suitable to track the patient position, size and/or shape.

In an example, the subject detection unit is configured to detect or extract positions or coordinates of anatomical landmarks of the subject and to detect an orientation of the subject based on the positions of the anatomical landmarks. The landmarks may be e.g. shoulders, neck, hip bones, etc.

In an example, the subject data is dimension data and/or phase data, wherein the dimension data comprise at least one of the group of the subject's shape, size, weight, body mass index, sex, age, position and orientation of the subject and/or at least a subject's landmark, and wherein the phase data comprise a heart cycle and/or a breathing cycle. The detection of the subject data may comprise an automatic detection and/or a manual input.

In an example, the X-ray pre-exposure control device may further comprise a patient positioning quality indication unit. This patient positioning quality indication unit may comprise a positioning quality sensor and a positioning quality indicator 172. The patient positioning quality indication unit may be used for improving the quality of X-ray or CXR examination by visual feedback of the quality of patient preparation to a radiographer prior to the X-ray exposure. The visual feedback of the quality indicator can be given for example with a traffic light with green symbolizing good positioning and red indicating that a re-positioning is necessary.

The position quality can be automatically derived from various types of the at least one positioning quality sensor or combinations of several positioning quality sensors of the same or different kind. The positioning quality sensor may be a contact sensor at e.g. an X-ray detector housing to measure a correct positioning of e.g. the subject's head, chin and/or arms. The positioning quality sensor may also be a force sensor e.g. on the ground to measure any imbalance in the subject's standing. The positioning quality sensor may also be an optical camera to track breathing of subject. The patient positioning quality indication unit may be connected with or attached to the X-ray unit to trigger an event when e.g. the radiographer presses an exposure button in a ‘poor quality’ state. The event can be an additional prompt to verify the exposure at the indicated poor positioning.

In an example, the subject model unit is configured to select the subject model based on at least one of the group of the subject's size, shape, weight, age, sex, thorax volume, distance between landmarks and/or the like from e.g. a database of pre-defined software models. The database may include models of different size (small/medium/large), age (child/adult) and sex (male/female). The selection procedure can be based on derived parameters from the body shape such as thorax volume, distance from left to right shoulder, distance from hip to shoulder and combinations thereof. The subject model can be e.g. a thorax model.

Furthermore, additional data on the subject may be collected by other sensors. For example, the subject might stand on a weight plate to measure its weight, from which a body mass index might be derived for further selection of the subject model and/or the image acquisition parameters. Furthermore, the breathing and heart cycle of the subject might be tracked to generate a 4D model of the subject.

The subject model unit refines the subject model based on the detected subject data into a refined subject model. The selected model may be refined by an adaptation to the landmarks, the subject's orientation, the subject's heart rate, the subject's breathing cycle and/or the like. The provision and/or the refinement of the subject model can be done automatically and/or manually. The adaptation step may comprise rigid and/or non-rigid transformations of the selected subject model.

In an example, the setting data of the X-ray unit to be used for exposing the subject is at least one of the group of position or orientation of an X-ray source, an X-ray detector, a focal spot or a collimator, exposure time, availability of a scatter grid, kVp and/or the like. The provision of the setting data can be done automatically and/or manually.

The setting data of the X-ray unit can be used to improve the simulation of the virtual X-ray projection. The virtual X-ray projection is computed through the refined or adapted subject model using the derived settings of the X-ray unit.

In an example, the setting data is a collimation parameter of the X-ray unit to be used for exposing a sub-region of the subject. In an example, the collimation parameter is a collimation window displayed by the display unit and the input unit is configured to interactively adjust the position, size and/or orientation of the collimation window.

In other words, the projected collimator boundaries can be computed in the virtual X-ray projection and the computed virtual X-ray projection image can be displayed on a viewing monitor to the radiographer. For an interactive adjustment of the collimators, a larger field-of-view may be displayed on the monitor together with an indication of the active collimated area. In this way, the radiographer can adjust the position of the collimators with direct visual feedback.

The device may be adjusted to raise a warning if automatically computed image measures indicate a poor image quality. Such indications may comprise for example a rotation of the subject or lung fields, which extend outside the collimated area. To this end, computer software may analyze the virtual X-ray projection for standard positioning quality criterions.

In an example, the processing unit is further configured to continuously recalculate the virtual X-ray projection based on the phase data, and the display unit is configured to continuously display the virtual X-ray projection based on the phase data. A dynamic virtual X-ray projection can be displayed indicating the breathing cycle of the subject to ensure the correct positioning of the subject in the breathing state (e.g. inspiration), in which the X-ray image is to be taken. In this way, the actual X-ray exposure can be triggered with the live feedback from the dynamic 2D virtual X-ray projection.

According to the present invention, also an X-ray imaging system is presented. The X-ray imaging system comprises the X-ray pre-exposure control device as described above and an X-ray unit configured to expose the subject to X-ray radiation. As described above, the X-ray pre-exposure control device comprises a subject detection unit, a subject model unit, an interface unit, a processing unit, and a display unit.

In an example, the X-ray imaging system further comprises a data base configured to provide several subject models to a subject model unit of the X-ray pre-exposure control device.

According to the present invention, also an X-ray imaging method is presented. It comprises the following steps, not necessarily in this order:

detecting subject data of the subject to be exposed,

providing a subject model,

refining the subject model based on the subject data into a refined subject model,

providing setting data of an X-ray unit to be used for exposing the subject, and

calculating a virtual X-ray projection based on the refined subject model and the provided setting data, and

displaying the virtual X-ray projection.

The X-ray imaging or X-ray pre-exposure control method allows computing the virtual X-ray projection from the actual subject using current settings (e.g. collimation, orientation, position) of the X-ray unit. As an example, above X-ray imaging method can be implemented as follows: Data on e.g. the shape and size of the subject are collected with for example optical cameras to measure the body shape. A subject model is automatically selected from the data base and adjusted to e.g. the subject's size. By using the current settings of the X-ray unit for a collimation and for a positioning of e.g. an X-ray source and an X-ray detector, a simulated virtual X-ray projection (CXR) is computed, on which the actual collimation window may be displayed. The virtual X-ray projection is displayed to e.g. a radiographer in order to decide whether the positioning of the subject and the collimation of the X-ray source are suitable for the current examination.

According to the present invention, also a computer program element is presented, wherein the computer program element comprises program code means for causing an X-ray pre-exposure control device and an X-ray imaging system as defined in the independent device claims to carry out the steps of the X-ray imaging method when the computer program is run on a computer controlling the of the devices.

It shall be understood that the X-ray pre-exposure control device, the X-ray imaging system, the X-ray imaging method, the computer program element for controlling such devices and the computer readable medium having stored such computer program element according to the independent claims have similar and/or identical preferred embodiments, in particular, as defined in the dependent claims. It shall be understood further that a preferred embodiment of the invention can also be any combination of the dependent claims with the respective independent claim.

These and other aspects of the present invention will become apparent from and be elucidated with reference to the embodiments described hereinafter.

### BRIEF DESCRIPTION OF THE DRAWINGS

Exemplary embodiments of the invention will be described in the following with reference to the accompanying drawings:

FIG. 1 shows schematically and exemplarily an embodiment of an X-ray imaging system according to the invention comprising an X-ray pre-exposure control device according to the invention.

FIG. 2 shows a schematic overview of steps of an exemplary embodiment of an X-ray imaging method according to the invention.

FIG. 3 shows on the left a tracked body shape with landmarks and orientation in front of an X-ray detector and on the right two thorax models of different size.

FIG. 4 shows on the left a field-of-view highlighted on a monitor and on the right an X-ray illumination of the adapted subject model for generation of the virtual X-ray projection.

FIG. 5 shows positioning quality sensors in form of contact sensors at a chin support and at grip handles on an X-ray detector housing.

FIG. 6 shows positioning quality sensors in form of force sensors on a ground plate that measure the weight on a right and on a left foot.

FIG. 7 shows a quality indicator display combined with an animated pictogram of an ideal breathing motion.

### DETAILED DESCRIPTION OF EMBODIMENTS

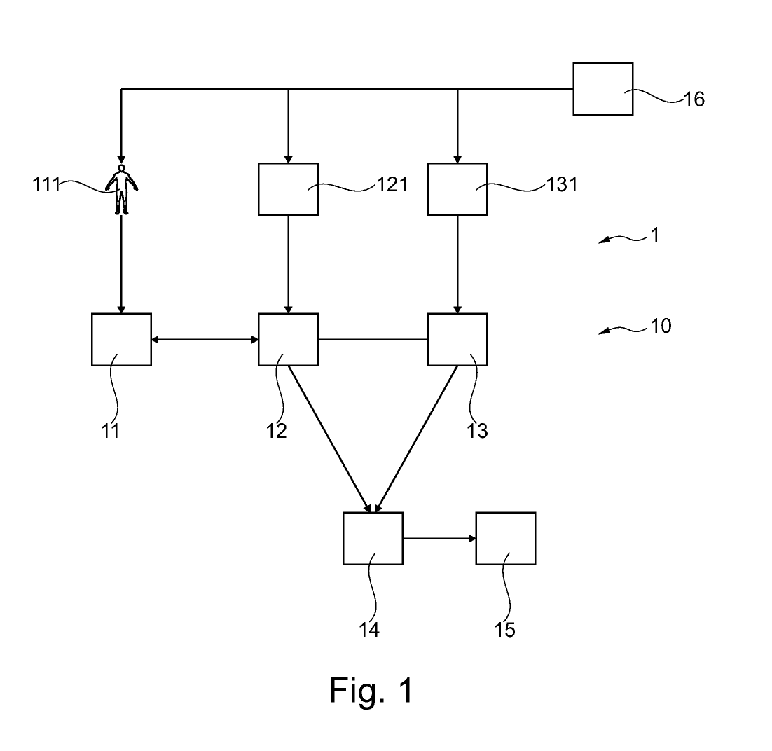

FIG. 1 shows schematically and exemplarily an embodiment of an X-ray imaging system 1 according to the invention. The X-ray imaging system 1 is configured for diagnostic imaging, in particular chest X-ray (CXR) examinations. The X-ray imaging system 1 comprises an X-ray unit 131 to expose a subject to X-ray radiation and an X-ray pre-exposure control device 10 to be explained in detail below. The X-ray imaging system 1 further comprises a data base 121 to provide a variety of subject models to a subject model unit 12 of the X-ray pre-exposure control device 10.

The X-ray pre-exposure control device 10 comprises a subject detection unit 11, a subject model unit 12, an interface unit 13, a processing unit 14, and a display unit 15. The X-ray pre-exposure control device 10 allows computing the virtual X-ray projection based on a refined subject model and current settings (e.g. collimation, orientation, position) of the X-ray unit 131. To this end, data on e.g. the shape and size of the subject 111 are collected with for example optical cameras to measure the body shape. Then, a subject model is automatically selected from the data base 121 and adjusted to e.g. the subject's size. By using the current settings of the X-ray unit 131 for a collimation and for a positioning of e.g. an X-ray source and an X-ray detector, a simulated virtual X-ray projection (CXR) is computed, on which the actual collimation window may be displayed. The virtual X-ray projection is displayed to e.g. a radiographer in order to decide whether the positioning of the subject 111 and the collimation of the X-ray source are suitable for the current examination.

In detail:

The subject detection unit 11 detects subject data of the subject 111 to be exposed. The subject data is dimension data and/or phase data. The dimension data comprise at least one of the group of the subject's shape, size, weight, body mass index, sex, age, position and orientation of the subject and/or at least a subject's landmark. The phase data comprise at least a heart cycle and/or a breathing cycle. The detection of the subject data may comprise an automatic detection and/or a manual input.

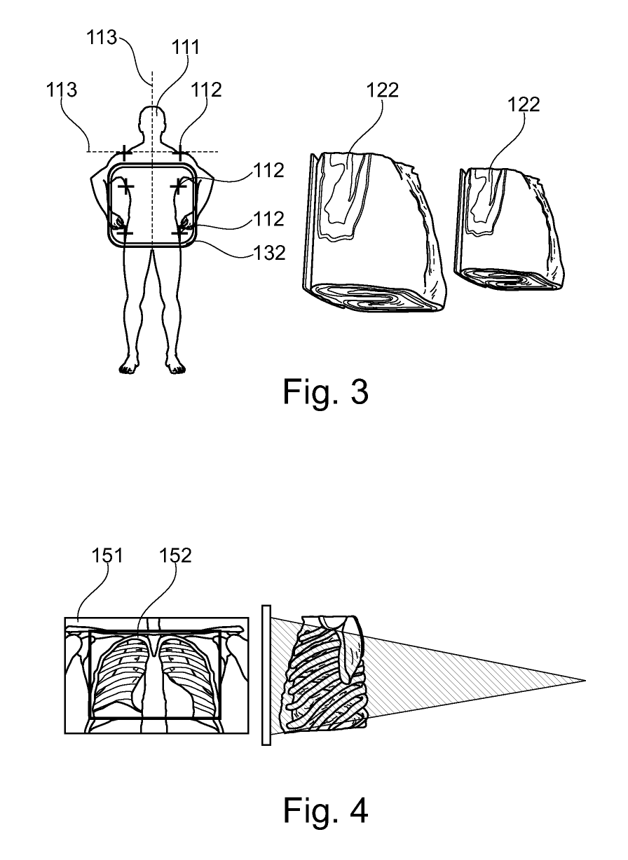

The subject detection unit 11 is at least one of the group of an optical, a time-of-flight, an infrared, an ultrasound, a radar camera or sensor, a weight sensor, a 3D depth sensor, a sensor sensing a breathing cycle, a sensor sensing a heart cycle, a millimeter wave sensor, a backscatter X-ray sensor and the like. The camera or sensor may be combined with infrared light. The subject detection unit 11 detects or extracts positions or coordinates of anatomical landmarks of the subject 111 and detects an orientation of the subject 111 based on the positions of the anatomical landmarks (see FIG. 3). The landmarks may be e.g. shoulders, neck, hip bones, etc.

The subject model unit 12 provides a subject model and refines the subject model based on the subject data into a refined subject model. The subject model unit 12 selects the subject model based on at least one of the group of the subject's size, shape, weight, age, sex, thorax volume, distance between landmarks and/or the like from e.g. a database of pre-defined software models. The database may include models of e.g. different size (small/medium/large), age (child/adult) and sex (male/female). The selection procedure can be based on derived parameters from the body shape such as thorax volume, distance from left to right shoulder, distance from hip to shoulder and combinations thereof. The subject model can be e.g. a thorax model.

The selected model is refined by an adaptation to the landmarks and/or the subject's orientation. The adaptation step may comprise rigid and/or non-rigid transformations of the selected subject model.

The interface unit 13 provides setting data of an X-ray unit 131 to be used for exposing the subject 111. The setting data of the X-ray unit 131 is at least one of the group of position or orientation of an X-ray source, an X-ray detector, a focal spot or a collimator, exposure time, availability of a scatter grid, kVp and/or the like. The provision of the setting data can be done automatically and/or manually.

The setting data of the X-ray unit 131 are used to improve the simulation of the virtual X-ray projection. The virtual X-ray projection is computed through the adapted subject model using the derived settings of the X-ray unit 131. The setting data is here a collimation parameter of the X-ray unit 131 to be used for exposing a sub-region of the subject. The collimation parameter is a collimation window (see FIG. 4) displayed by the display unit 15 and the input unit 16 is configured to allow an interactive adjustment of the position, size and/or orientation of the collimation window.

In other words, the projected collimator window boundaries are computed in the virtual X-ray projection and the computed virtual X-ray projection image are here displayed on a viewing monitor to the radiographer. For an interactive adjustment of the collimators, a larger field-of-view may be displayed on the monitor together with an indication of the active collimated area. In this way, the radiographer can adjust the position of the collimators with direct visual feedback.

The processing unit 14 calculates a virtual X-ray projection based on the refined subject model and the provided setting data. The processing unit 14 further continuously recalculates the virtual X-ray projection based on the phase data, and the display unit 15 continuously displays the virtual X-ray projection based on the phase data. The display unit 15 displays the virtual X-ray projection.

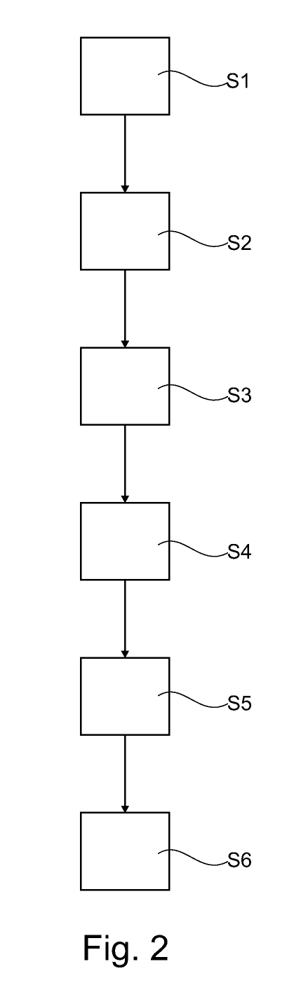

FIG. 2 shows a schematic overview of steps of an exemplary embodiment of an X-ray imaging method according to the invention. The method comprises the following steps, not necessarily in this order:

detecting subject data of the subject 111 to be exposed.

providing a subject model.

refining the subject model based on the subject data into a refined subject model.

providing setting data of an X-ray unit 131 to be used for exposing the subject.

calculating a virtual X-ray projection based on the refined subject model and the provided setting data.

displaying the virtual X-ray projection.

These steps will be explained in further detail below. In the first step S1, the subject or patient is tracked with for example optical cameras, time-of-flight cameras, or 3D depth sensors in combination with infrared light. From the resulting body shape model or subject model, coordinates of landmarks of the subject such as shoulders, neck, hip bones are extracted and used to compute the orientation of the subject's body.

FIG. 3 shows hereto on the left a tracked body shape of a subject 111 with landmarks 112 marked by crosses. The displayed body shape also shows the orientation of the subject 111 marked by the dotted lines 113 in front of the X-ray detector 132.

From the tracked body shape, a thorax model is selected in a second step S2 from a database of pre-defined software models. The database here includes models of different size (small/medium/large), age (child/adult) and sex (male/female). FIG. 3 shows hereto on the right thorax models 122 of different size. The selection procedure is here based on derived parameters from the body shape of step S1 such as thorax volume, distance from left to right shoulder, distance from hip to shoulder and combinations thereof.

Further, additionally data on the subject 111 may be collected with other sensors. For example, the subject 111 might stand on a weight plate (not shown) to measure its weight, from which a body mass index might be derived for further selection of the subject model and the image acquisition parameters. Furthermore, the breathing and heart cycle of the subject 111 might be tracked to generate a 4D model of the subject.

In the third step S3, the selected model is refined by adaptation to the landmarks 112 and the subject's orientation as generated in step Sl. The adaptation step S3 may comprise rigid and non-rigid transformations of the selected subject model.

A viewport of the X-ray system is retrieved in step S4, i.e. the position of the focal spot, the position and orientation of the detector unit and the collimator positions are derived from the system. Further, acquisition settings such as kVp, exposure time, availability of a scatter grid may be derived to improve the following simulation of the virtual X-ray projection.

The virtual X-ray projection is computed in step S5 through the adapted subject model using the derived settings of the X-ray unit 131. Furthermore, the projected collimator boundaries are computed in the virtual X-ray projection.

As shown in FIG. 4, left, in step S6, the computed virtual X-ray projection 151 image is displayed on a viewing monitor to a radiographer. For interactive adjustment of the collimators, in FIG. 4, left, a field-of-view 152 is highlighted on the monitor. Also an indication of the active collimated area can be displayed. In this way, the radiographer can adjust the position of the collimators with direct visual feedback. FIG. 4, right, shows an X-ray illumination of the adapted subject model for generation of the virtual X-ray projection 151.

The system may be adjusted to raise a warning if automatically computed image measures indicate a poor image quality. Such indications may comprise for example a rotation of the subject or lung fields, which extend outside the collimated area. To this end, a computer software may analyze the virtual X-ray projection 151 for standard positioning quality criterions. In another embodiment of this invention, a dynamic virtual X-ray projection 151 is displayed indicating the breathing cycle of the subject to ensure the correct positioning of the subject in the breathing state (e.g. inspiration), in which the X-ray image is to be taken. In this way, the actual X-ray exposure can be triggered with the live feedback from the dynamic 2D virtual X-ray projection 151.

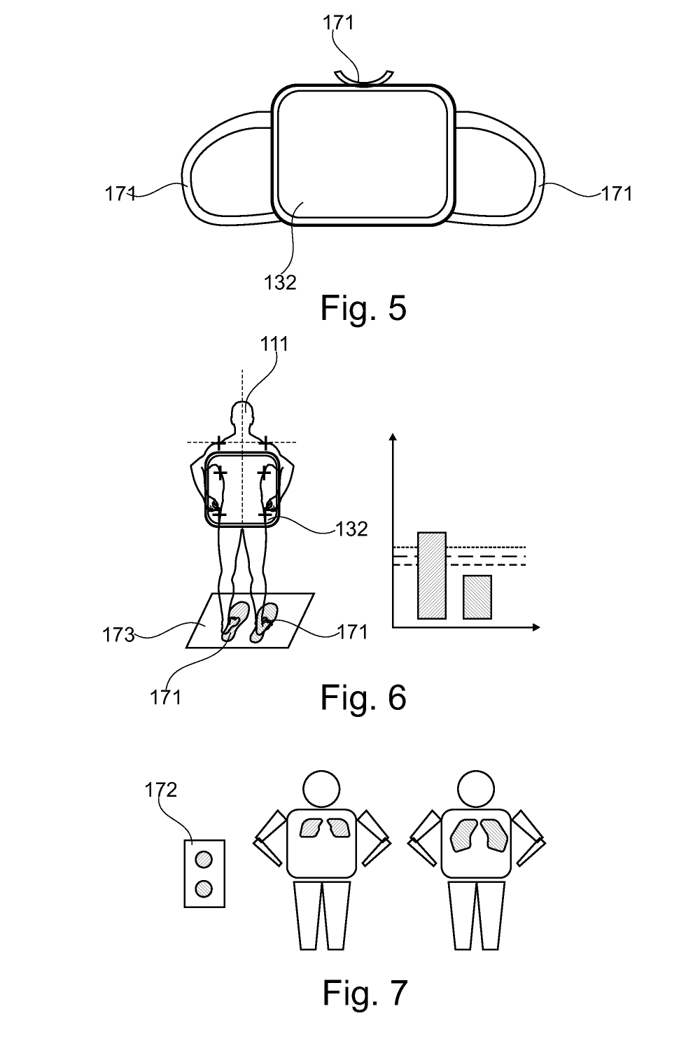

The X-ray pre-exposure control device 10 here further comprises a patient positioning quality indication unit. This patient positioning quality indication unit comprises a positioning quality sensor 171 (see FIGS. 5 to 7) and a positioning quality indicator 172 (see FIG. 7). The patient positioning quality indication unit may be used for improving the quality of X-ray or CXR examination by visual feedback of the quality of patient preparation to a radiographer prior to the X-ray exposure. The visual feedback of a quality indicator can be given for example with a traffic light with green symbolizing good positioning and red indicating that a re-positioning is necessary (see FIG. 7). The quality indicator can also be used to prevent the X-ray system to be used, when the positioning is not good enough, or to trigger an event in the X-ray unit 131 to display an additional prompt to verify the exposure in the non-optimal positioning. In this case, the X-ray unit 131 is blocked from doing an X-ray exposure if the quality indicator is below a pre-defined quality threshold, otherwise the X-ray unit 131 is set to a state, in which X-ray examinations are possible.

The position quality can be automatically derived from various types of at least one positioning quality sensor 171 or combinations of several positioning quality sensors 171 of the same or different kind. The positioning quality sensors 171 shown in FIG. 5 are contact sensors at a chin support (above) and at grip handles on an X-ray detector 132 housing measuring if the subject's chin rests in the support and if the subject has turned his arms towards the grip handle. In this way, a proper and correct positioning of the subject is facilitated. The positioning quality sensors 171 send information on contact/no contact to the quality indicator algorithm. The chin contact sensor may additionally be equipped with a force sensor. Measuring continuously the applied force on the chin sensor and sending this continuous information to the quality indicator algorithm enables to check whether the subjects is standing still in front of the detector.

The positioning quality sensors 171 shown in FIG. 6, left, are force sensors on a ground plate 173 to measure the weight on the right and on the left foot of the subject 111. The positioning quality sensors 171 are indicated with a drawing on the ground plate 173. As shown in FIG. 6, right, the positioning quality sensors 171 send a continuous signal on the weight distribution to the quality indicator algorithm to compute whether the subject 111 is standing balanced in front of the X-ray detector 132 to avoid any rotations of the subject 111.

The positioning quality sensors 171 can also be optical cameras or electromagnetic sensors to track the subject shape and to send an image to the quality indicator algorithm to analyze a centered subject positioning. These sensors may additionally measure the state of the breathing cycle of the patient.

The information of all positioning quality sensors 171 can be combined into one quality indicator value, for example by increasing a counter if a respective positioning quality sensor 171 provides a signal above a sensor-specific threshold. The signal is regularly updated from the continuous data of the positioning quality sensors 171. If the overall quality indicator value is above a final quality value threshold, a visual signal is displayed to the radiographer.

As shown in FIG. 7, left, the visual feedback of the positioning quality indicator 172 is given with a traffic light with green symbolizing good positioning and red indicating that a re-positioning is necessary. Here, the quality indicator display is combined with displaying an animated pictogram of an ideal breathing motion of the subject. A visual signal indicates the quality of positioning and/or the breathing state, i.e. exhale (FIG. 7, center) and inhale (FIG. 7, right). The subject or patient is asked to breath according to the displayed animated breathing state representation. For an intended exposure in the inhale state, the positioning quality indicator 172 is set to low quality (red light) in the exhale state of the animation and to good quality (in case all other sensors report a good positioning) in the inhale state of the animation. In another embodiment, the actual breathing state of the patient is measured with an optical camera and used for computing the positioning quality indicator 172.

In another exemplary embodiment of the present invention, a computer program or a computer program element is provided that is characterized by being adapted to execute the method steps of the method according to one of the preceding embodiments, on an appropriate system.

The computer program element might therefore be stored on a computer unit, which might also be part of an embodiment of the present invention. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfil the procedure of an exemplary embodiment of the method as described above.

According to a further exemplary embodiment of the present invention, a computer readable medium, such as a CD-ROM, is presented wherein the computer readable medium has a computer program element stored on it, which computer program element is described by the preceding section.

A computer program may be stored and/or distributed on a suitable medium, such as an optical storage medium or a solid state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfil the functions of several items re-cited in the claims. The mere fact that certain measures are re-cited in mutually different dependent claims does not indicate that a combination of these measures cannot be used to advantage. Any reference signs in the claims should not be construed as limiting the scope.

1. An X-ray pre-exposure control device, comprising:
a subject detection unit,
a subject model unit,
an interface unit,
a processing unit, and
a display unit,
wherein the subject detection unit is configured to detect subject data of the subject to be exposed,
wherein the subject model unit is configured to provide a subject model and to refine the subject model based on the subject data into a refined subject model,
wherein the interface unit is configured to provide setting data of an X-ray unit to be used for exposing the subject,
wherein the processing unit is configured to calculate a virtual X-ray projection based on the refined subject model and the provided setting data, and
wherein the display unit is configured to display the virtual X-ray projection.

2. X-ray pre-exposure control device according to claim 1, further comprising an input unit configured to adjust the subject, the subject model and/or the setting data of the X-ray unit, wherein the processing unit is further configured to recalculate the virtual X-ray projection based on this adjustment, and wherein the display unit is configured to display the virtual X-ray projection based on this adjustment.

3. X-ray pre-exposure control device according claim 2, wherein the setting data is a collimation parameter of the X-ray unit to be used for exposing a sub-region of the subject.

4. X-ray pre-exposure control device according to claim 3, wherein the collimation parameter is a collimation window displayed by the display unit and wherein the input unit is configured to interactively adjust the position, size and/or orientation of the collimation window.

5. X-ray pre-exposure control device according to claim 1, wherein the subject detection unit is configured to detect positions of anatomical landmarks of the subject and to detect an orientation of the subject based on the positions of the anatomical landmarks.

6. X-ray pre-exposure control device according to claim 1, wherein the subject detection unit at least one of the group of an optical, an infrared, an ultrasound, a radar camera or sensor, a weight sensor, a depth sensor, a sensor sensing a breathing cycle, a sensor sensing a heart cycle, a millimeter wave sensor and a backscatter X-ray sensor.

7. X-ray pre-exposure control device according to claim 6, wherein the subject data is dimension data and/or phase data,
wherein the dimension data comprise at least one of the group of the subject's shape, size, position and orientation,
wherein the phase data comprise a heart cycle and/or a breathing cycle, and
wherein the processing unit is configured to continuously recalculate the virtual X-ray projection based on the phase data, and wherein the display unit is configured to continuously display the virtual X-ray projection based on the phase data.

8. X-ray pre-exposure control device according to claim 1, further comprising a patient positioning quality indication unit comprising a positioning quality sensor configured to detect a subject's positioning relative to an X-ray unit.

9. X-ray pre-exposure control device according to claim 8, wherein the positioning quality sensor comprise at least one of the group of a contact sensor, a force sensor, and an optical camera, wherein the latter is configured to track a subject's breathing.

10. X-ray pre-exposure control device according to claim 1, wherein the subject model unit is configured to select the subject model based on at least one of the group of the subject's size, weight, age, sex, thorax volume and distance between landmarks.

11. X-ray pre-exposure control device according to claim 1, wherein the setting data of the X-ray unit to be used for exposing the subject is at least one of the group of position or orientation of an X-ray source, detector, focal spot or collimator, exposure time, availability of a scatter grid and kVp.

12. An X-ray imaging system, comprising:
an X-ray pre-exposure control device according to claim 1, and
an X-ray unit,
wherein the X-ray unit is configured to expose the subject to X-ray radiation.

13. An X-ray imaging method with pre-exposure controlling, comprising the following steps:
detecting subject data of the subject to be exposed,
providing a subject model,
refining the subject model based on the subject data into a refined subject model,
providing setting data of an X-ray unit to be used for exposing the subject,
calculating a virtual X-ray projection based on the refined subject model and the provided setting data, and
displaying the virtual X-ray projection.

14. A computer program element for controlling a device or system according to claim 1, which, when being executed by a processing unit, is adapted to perform the method steps.

15. A computer readable medium having stored the computer program element of claim 14.
